(19) United States
(12) Patent Application Publication
(10) Pub. No.: US 2002/0086291 A1
(43) Pub. Date: Jul. 4, 2002

(54) Methods and reagents for analysis of rna structure and function

(76) Inventors: David P. Hornby, Cheshire (GB); Mark Dickman, Sheffield (GB)

Correspondence Address: WILLIAM B. WALKER, TRANSGENOMIC, INC., 2032 CONCOURSE DRIVE, SAN JOSE, CA, 95131, US

(21) Appl. No.: 09/727,138

(22) Filed: Nov. 29, 2000

(51) Int. Cl. C12Q 1/68

(52) U.S. Cl. 435/6

(57) ABSTRACT

The present invention describes a method for identifying bases in an RNA sequence that are relatively inaccessible to solvent as the result of intramolecular and/or intermolecular interactions. Embodiments of the invention can be used to evaluate the three-dimensional structure of an RNA molecule or the interactions between an RNA molecule and a molecule capable of binding to RNA, such as another nucleic acid or an RNA-binding protein. The invention involves contacting an RNA molecule with a cleavage reagent capable of partially hydrolyzing said RNA molecule, wherein said partial hydrolysis is attenuated in a region of said RNA molecule that is relatively inaccessible to solvent; and separating and detecting the cleaved RNA by IP-RP-HPLC, wherein the absence of cleavage events in a region of the RNA indicates that said region is relatively inaccessible to solvent. The separation medium is preferably substantially free of multivalent cations capable of interfering with polynucleotide separations.

### FIELD OF THE INVENTION

The present invention is directed to methods and materials useful for characterizing intramolecular and intermolecular RNA interactions. The invention can be used to analyze secondary and tertiary interactions of RNA structures.

### BACKGROUND OF THE INVENTION

The ribonucleic acids (“RNAs”) are an important family of biological macromolecules involved in various aspects of translating the information encoded by the genome into the corresponding gene products. RNAs are distinguished by their unique diversity of function; not only do they play a major role in protein translation (i.e., the tRNAs, rRNAs and mRNAs), but they are also capable of acting as primary stores of genetic information (e.g., viral RNAs) and as biological catalysts (e.g., catalytic RNA, or “ribozymes”), functions traditionally associated with DNA and proteins, respectively.

RNA molecules are usually single-stranded, but most have self-complementary regions that form hairpin structures, and some have well-defined tertiary structures. The biological functions of RNA molecules, such as their ability to catalyze chemical reactions or to specifically associate with proteins or other nucleic acids, is dependent upon this three dimensional structure. Thus, to a large extent, an understanding of the biological function of RNA requires unraveling the structure and folding pathways of these molecules.

One technique that has been employed to elucidate RNA structure and folding pathways is “RNA footprinting,” a technique that is analogous to DNA footprinting. Both techniques identify regions of the molecule that are inaccessible to solvent. However, while DNA footprinting is normally used to identify protein-binding regions, RNA footprinting has typically been used to identify regions where solvent is excluded due to secondary and tertiary interactions, from which structural information is inferred.

RNA footprinting is generally accomplished by treating an RNA molecule with an agent capable of cleaving the phosphodiester bonds linking the ribonucleotides in a relatively non-specific, sequence-independent manner. The extent of cleavage is assessed, traditionally by gel electrophoresis, and regions of restricted solvent accessibility are identified by reduced levels of cleavage. Solvent accessibility in a region indicates that the region is involved in some interaction that is protecting the molecule from the cleavage reaction. For example, Hampel and colleagues reported the use of hydroxyl radical footprinting to define the solvent-protected core of the hairpin ribozyme-substrate complex (Hampel et al. (1998) Biochemistry 37:14672-82).

A better understanding of the structural and functional properties of RNAs will be critical in the development of new RNA-based therapeutics and therapeutic compounds that target RNA. Proposed RNA-related therapeutics include antisense oligonucleotides and other molecules that affect transcription and transcript levels, ribozymes that target RNA molecules involved in genetic diseases and other diseases such as HIV infection, cancer and arthritis, and small molecules designed to modulate the interaction between RNA and RNA binding proteins. For example, in a gene therapy approach to HIV infection ribozymes have been used to destroy HIV RNA molecules and make cells resistant to the effects of HIV infection.

Most of the currently available RNA footprinting protocols (and DNA footprinting protocols) rely on the use of radiolabeled-oligonucleotides and gel electrophoresis. The use of radioactivity requires that special safety precautions be taken, and the disposal of the radioactive waste that necessarily results from these methods can be inconvenient and expensive. Moreover, the use of sequencing gels is inconvenient, hazardous, time consuming, and can yield inconsistent results in the hands of different technicians. Gel banding patterns are also notoriously difficult to quantify and interpret. Sequencing gels produced in different laboratories are often difficult to compare quantitatively due to the reproducibility problems inherent to pouring and running gels. The bands representing distinct polynucleotide populations are often curved rather than straight, their mobility and shape can change across the width of the gel, and lanes and bands can mix with each other. These inaccuracies typically stem from the lack of uniformity and homogeneity of the gel bed, electroendosmosis, thermal gradient and diffusion effects, as well as host of other factors. Inaccuracies of this sort can lead to serious distortions and inaccuracies in the display of the separation results. In addition, the band display data obtained from gel electrophoresis separations is not quantitative or accurate because of the uncertainties related to the shape and integrity of the bands. True quantitation of linear band array displays produced by gel electrophoresis separations cannot be achieved, even when the linear band arrays are scanned with a detector and the resulting data are integrated, because the linear band arrays are scanned only across the center of the bands. Since the detector only sees a small portion of any given band and the bands are not uniform, the results produced by the scanning method are not accurate and can even be misleading. Furthermore, methods for visualizing gel electrophoretic separations, such as staining or autoradiography, tend to be cumbersome and time consuming. Furthermore, gel electrophoresis is difficult to automate and to practice in a high-throughput manner.

It would thus be desirable to have available improved methods for RNA footprinting that do not rely on the use of radioactive labels or gel electrophoresis. This would advance our understanding of RNA structure and the nature of specific interactions between RNA and other molecules, e.g., other nucleic acids and RNA binding proteins, which will in turn facilitate the development of novel RNA-based therapeutics. By providing such improved methods, the present invention represents a valuable contribution to the fields of molecular biology and medicine.

### SUMMARY OF THE INVENTION

The present invention provides novel methods and reagents useful for the analysis of RNA.

In one aspect, the invention entails determining bases in an RNA sequence that are relatively inaccessible to solvent as the result of intramolecular and/or intermolecular interactions.

In preferred embodiments, the invention can be used to evaluate the three-dimensional structure (i.e., secondary and/or tertiary structure) of an RNA molecule; the interactions between an RNA molecule and a molecule capable of binding to RNA, such as another nucleic acid or an RNA-binding protein. In a particularly preferred embodiment, the invention can be used to evaluate the structural nature of a complex involving a ribozyme and its substrate.

In one aspect, the invention provides a method for analyzing the structural properties of an RNA molecule that comprises contacting said RNA molecule with a cleavage reagent capable of partially hydrolyzing said RNA molecule, wherein said partial hydrolysis is attenuated in a region of said RNA molecule that is relatively inaccessible to solvent; and separating and detecting the cleaved RNA by IP-RP-HPLC, wherein the absence of cleavage events in a region of the RNA indicates that said region is relatively inaccessible to solvent.

In a preferred embodiment of the invention, IP-RP-HPLC employs a separation medium that is substantially free of multivalent cations capable of interfering with polynucleotide separations.

In an aspect of the invention, the separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a nonpolar hydrocarbon or substituted hydrocarbon group, wherein said surfaces are non-polar.

In another aspect of the invention, the separation medium comprises polymer beads having an average diameter of 0.5 to 100 microns, said beads being unsubstituted polymer beads or polymer beads substituted with a moiety selected from the group consisting of hydrocarbon having from one to 1,000,000 carbons.

In yet another aspect of the invention the separation medium comprises a monolith.

Preferred embodiments of the invention employ a separation medium that has been subjected to acid wash treatment to remove any residual surface metal contaminants and/or has been subjected to treatment with a multivalent cation binding agent.

In one aspect of the invention, the IP-RP-HPLC employs a mobile phase comprising a solvent selected from the group consisting of alcohol, nitrile, dimethylformamide, tetrahydrofuran, ester, ether, and mixtures of one or more thereof, preferably acetonitrile.

In yet another aspect of the invention, said mobile phase comprises a counterion agent selected from the group consisting of lower alkyl primary amine, lower alkyl secondary amine, lower alkyl tertiary amine, lower trialkylammonium salt, quaternary ammonium salt, and mixtures of one or more thereof.

In a preferred embodiment of the invention, the counterion agent is selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyidiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof. Tetrabutylammonium acetate and triethylammonium acetate are particularly preferred counterion agent.

In preferred embodiments of the invention, the counterion agent includes an anion, said anion is selected from the group comprising acetate, carbonate, phosphate, sulfate, nitrate, propionate, formate, chloride, and bromide.

In particularly preferred embodiments of the invention, the detection is achieved using Matched Ion Polynucleotide Chromatography.

In one aspect the RNA molecule is detectably labeled, preferably by means of a fluorescent label. In a preferred embodiment of the invention the label is selected from the group consisting of FAM, JOE, TAMRA, ROX, HEX, TET, Cy3, and Cy5.

In a preferred embodiment of the invention the RNA cleavage reagent is a hydroxyl radical. In a particularly preferred embodiment of the invention the hydroxyl radical is generated using Fe(EDTA)²⁻.

In another aspect of the invention the RNA cleavage reagent is a nuclease, preferably an RNase.

In a preferred embodiment of the invention, the IP-RP-HPLC separation is phased by running a parallel RNA cleavage reaction, preferably an RNA sequencing reaction.

In one aspect, the RNA molecule includes region that is relatively inaccessible to solvent owing to intramolecular interactions.

In another aspect, the RNA molecule includes region that is relatively inaccessible to solvent owing to intermolecular interactions.

In a preferred embodiment of the invention, the method is used to characterize the three-dimensional structure of an RNA molecule.

In another preferred embodiment of the invention, the method is used to characterize the interaction between a ribozyme and its substrate.

In still another preferred embodiment of the invention, the method is used to characterize the interaction between an RNA molecule and an RNA-binding protein.

### BRIEF DESCRIPTION OF THE FIGS.

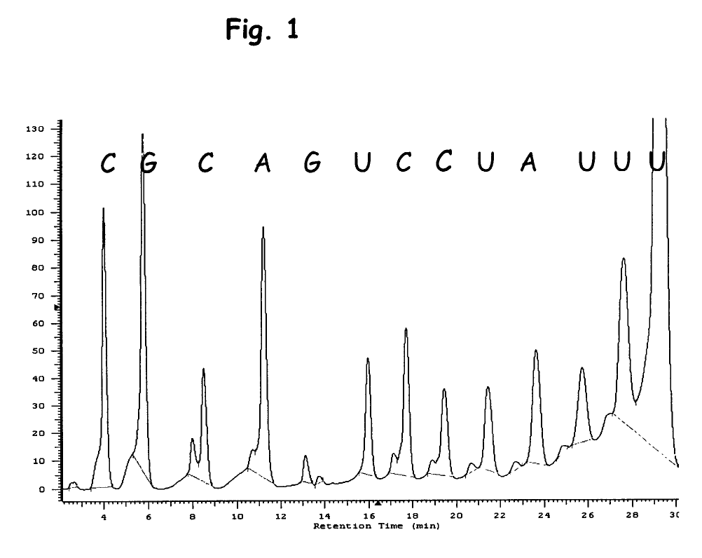

FIG. 1 shows the chromatogram generated by base catalyzed hydrolysis of the fluorescently labeled substrate strand of the hairpin ribozyme, as described in Example 1.

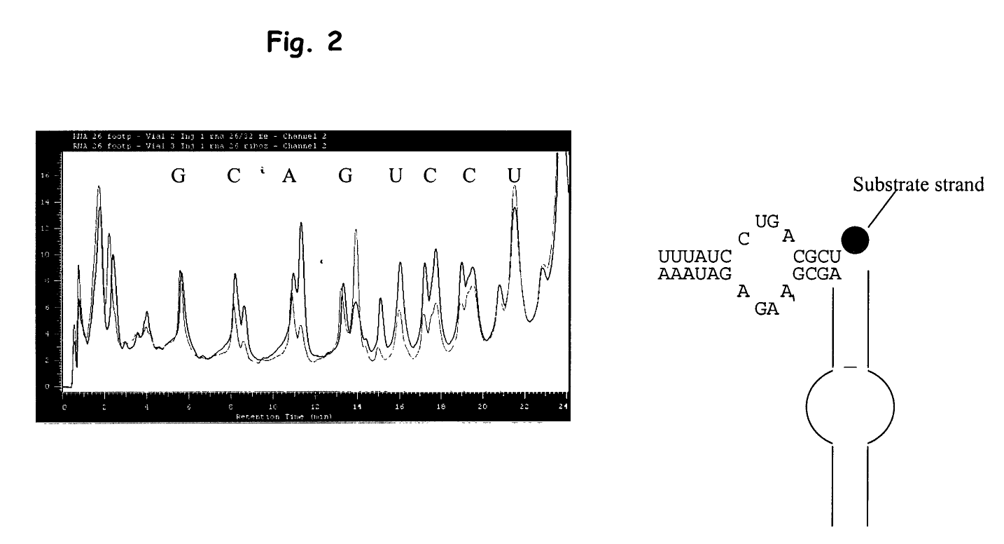

FIG. 2 shows the footprint of the ribozyme substrate strand in the docked ribozyme complex, compared to the cleavage of the substrate strand in free solution, as described in Example 2.

### DETAILED DESCRIPTION OF THE INVENTION

As described above, the need exists for an economical, high-throughput method for RNA footprinting that avoids the limitations inherent in currently available methods. The present invention provides novel methods and reagents that satisfy this need.

It is therefore an object of the instant invention to provide improved methods and reagents for determining a position in an RNA sequence that having restricted solvent accessibility.

It is a further object of the present invention to provide improved methods and reagents for evaluating the three-dimensional structure (i.e., secondary and/or tertiary structure) of an RNA molecule.

It is a further object of the present invention to provide improved methods and reagents for evaluating the intra-molecular interactions in an RNA molecule.

It is a further object of the present invention to provide improved methods and reagents for evaluating the interactions between an RNA molecule and a molecule capable of binding to RNA, such as another nucleic acid or an RNA-binding protein.

It is a further object of the present invention to provide improved methods and reagents for evaluating a complex between a ribozyme and its substrate.

Practice of the instant invention can entail a variety of techniques and methods known to one of skill in the art. Such methods are widely available and provided, for example, in Molecular Cloning: a Laboratory Manual: 2nd edition, 3 Volumes, Sambrook et al, 1989, Cold Spring Harbor Laboratory Press (or later editions of the same work) or Current Protocols in Molecular Biology, Second Edition, Ausubel et al. eds., John Wiley & Sons, 1992.

In one aspect, the methods and reagents of the instant invention can be used to evaluate and/or characterize the three-dimensional structure of an RNA molecule, or a complex involving an RNA molecule. In a preferred embodiment, the RNA molecule is capable of functioning as a catalyst. Examples of ribozymes include the hairpin ribozyme, described by Hampel et al., supra.

In another aspect, the methods and reagents of the instant invention can be used to evaluate and/or characterize the interaction between an RNA molecule and another molecule capable of binding to the RNA molecule. In a preferred embodiment, the other RNA-binding molecule is an RNA binding protein. RNA binding proteins (RBP) appear to mediate the processing of pre-mRNAs, the transport of mRNA from the nucleus to the cytoplasm, mRNA stabilization, the translational efficiency of mRNA, and the sequestration of some mRNAs. Recent studies have identified several RNA-binding motifs in a diversity of RBPs. The most common RNA binding protein motifs are the RNP motif, Arg-rich motif, RGG box, KH motif and double-stranded RNA-binding motif (for review see Burd and Dreyfuss, Science 265:615-621 (1994)). These motifs recognize both sequence and structure dependent RNA elements. In the case of the double-stranded RNA-binding motif, sequence recognition is unimportant. However, in addition to the double stranded structure, a positional effect for the double-stranded RNA may play a role in recognition (Bass, Nucleic Acids Symposium 33:13-15 (1995)) and some of these proteins may also require binding to Z-DNA prior to their activity on the double-stranded RNA (Herbert et al., Proc. Natl. Acad. Sci. USA 92:7550-7554 (1995)). In addition, other RNA binding proteins, such as AUBF (Malter, Science 246:664-666 (1989)) are likely to bind in a structureindependent manner.

RNA molecules for use in the disclosed method can be part of a crude cellular or nuclear extract, partially purified, or extensively purified. RNA molecules can also be made by in vitro transcription or by direct synthesis. RNA molecules can be used either in isolation or in combination with one or more other RNA molecules. In a preferred embodiment the RNA molecule is produced in vitro.

RNA molecules can be prepared using known methods for preparing cellular extracts and for purifying RNA. Methods for preparing extracts containing RNA molecules are described in, for example, Sambrook et al., and Ausubel et al. Individual RNA molecules can also be produced recombinantly using known techniques, by in vitro transcription, and by direct synthesis. For recombinant and in vitro transcription, DNA encoding RNA molecules can be obtained from known clones, by synthesizing a DNA molecule encoding an RNA molecule, or by cloning the gene encoding the RNA molecules. Techniques for in vitro transcription of RNA molecules and methods for cloning genes encoding known RNA molecules are described by, for example, Sambrook et al. Synthetic RNAs can be prepared, for example, on an Applied Biosystems (Foster City, Calif.) 392 DNA/RNA synthesizer using standard phosporamidite chemistry.

In a preferred embodiment, particularly where quantitation is desired, the RNA is detectably labeled, preferably by end-labeling. For example, the substrate can be end-labeled using T4 polynucleotide kinase and [γ-³²P]ATP, or with other reagents, such as biotin, digoxigenin, fluorescein or another fluorophore, depending on the particular detection and quantification system to be employed. Generally, labels known to be useful for nucleic acids can be used to label RNA molecules.

In a particularly preferred embodiment of the invention, the RNA is labeled with a fluorescent group. Non-limiting examples of fluorescent groups suitable for use with the instant invention include 5-carboxyfluorescein (5-FAM), 6-carboxyfluorescein (6-FAM), 2′,7′-dimethoxy-4′,5′-dichloro-6-carboxyfluorescein (JOE), N,N,N′-N-tetramethyl-6-carboxy rhodamine (TAMRA), 6-carboxy-X-rhodamine (ROX), 4,7,2′,4′,5′,7′-hexachloro-6-carboxy-fluorescein (HEX-1), 4,7,2′,4′,5′,7′-hexachloro-5-carboxy-fluorescein (HEX-2), 2′,4′,5′,7′-tetrachloro -5-carboxy-fluorescein (ZOE), 4,7,2′,7′-tetrachloro-6-carboxy-fluorescein (TET-1), 1), 1′,2′,7′,8′-dibenzo-4,7-dichloro-5-carboxyfluorescein (NAN-2), and 1′,2′,7′,8′-dibenzo-4,7-dichloro-6-carboxyfluorescein, fluorescein and fluorescein derivatives, Rhodamine, Cascade Blue, Alexa₃₅₀, Alexa₄₈₈, , phycoerythrin, allo-phycocyanin, phycocyanin, rhodamine, Texas Red, EDANS, BODIPY dyes such as BODIPY-FL and BODIPY-TR-X, tetramethylrhodamine, Cy3 and Cy5, 5,6-carboxyfluorescein, fluorescein mono-derivatized with a linking functionality at either the 5 or 6 carbon position, including fluorescein-5-isothiocyanate, fluorescein-6-isothiocyanate (the -5- and -6-forms being referred to collectively as FITC), fluorescein-5-succinimidylcarboxylate, fluorescein-6-succinimidylcarboxylate, fluorescein-5-iodoacetamide, fluorescein-6-iodoacetamide, fluorescein-5-maleimide, and fluorescein-6-maleimide; , 2′,7′-dimethoxy-4′,5′-dichlorofluorescein mono-derivatized with a linking functionality at the 5 or 6 carbon position, including 2′,7′-dimethoxy-4′,5′-dichlorofluorescein-5-succinimidylcarboxylate and 2′,7′-dimethoxy-4′,5′-dichlorofluoescein-6-succinimidylcarboxylate (the -5- and -6-forms being referred to collectively as DDFCS), tetramethyirhodamine mono-derivatized with a linking functionality at either the 5 or 6 carbon position, including tetramethylrhodamine-5-isothiocyanate, tetramethylrhodamine-6-isothiocyanate (the -5- and -6-forms being referred to collectively as TMRITC), tetramethylrhodamine-5-iodoacetamide, tetramethylrhodamine-6-iodoacetamide, tetramethylrhodamine-5-succinimidylcarboxylate, tetramethylrhodamine-6-succinimidylcarboxylate, tetramethylrhodamine-5-maleimide, and tetramethylrhodamine-6-maleimide, rhodamine X derivatives having a disubstituted phenyl attached to the molecule's oxygen heterocycle, one of the substituents being a linking functionality attached to the 4′or 5′carbon (IUPAC numbering) of the phenyl, and the other being a acidic anionic group attached to the 2′carbon, including Texas Red (tradename of Molecular Probes, Inc.), rhodamine X-5-isothiocyanate, rhodamine X-6-isothiocyanate, rhodamine X-5-iodoacetamide, rhodamine X-6-iodoacetamide, rhodamine X-5-succinimidylcarboxylate, rhodamine X-6-succinimidylcarboxylate, rhodamine X-5-maleimide, and rhodamine X-6-maleimide.

Fluorescent labels can be attached to a polynucleotide using standard procedures, e.g. for a review see Haugland, “Covalent Fluorescent Probes,” in Excited States of Biopolymers, Steiner, Ed. (Plenum Press, New York, 1983), incorporated by reference herein in its entirety. In a preferred embodiment of the invention, a fluorescent group can be covalently attached to a desired primer by reaction with a 5′-amino-modified oligonucleotide in the presence of sodium bicarbonate and dimethylformamide, as described in U.S. Pat. application No. 09/169,440. Alternatively, the reactive amine can be attached by means of the linking agents disclosed in U.S. Pat. No. 4,757,141.

Alternatively, covalently tagged primers can be obtained commercially (e.g., from Midland Certified Reagent, Co.). Fluorescent dyes are available form Molecular Probes, Inc. (Eugene, Oreg.), Operon Technologies, Inc., (Alameda, Calif.) and Amersham Pharmacia Biotech (Piscataway, N.J.), or can be synthesized using standard techniques. Fluorescent labeling is described in U.S. Pat. No. 4,855,225.

The reaction should be conducted under conditions that result in cleavage of unprotected, solvent-accessible RNA, but that do not interfere with or otherwise disrupt RNA secondary or tertiary structure, or other inter- or intra-molecular interactions that would occur normally under the conditions of interest, especially physiological conditions. Hence, it is preferable that the reaction solution is pH buffered, with a pH that is preferably between 4 and 10, more preferably between 6 and 8, and most preferably around 7.0 to 7.4. The reaction should be conducted at a temperature that does not disrupt the native structure of the RNA and any inter- or intra-molecular interactions, which can vary depending upon the specific molecule or molecules being studied. In many instances room temperature is suitable.

The assay buffer used depends on the nature of the RNA and, if applicable, any binding molecules, the questions being addressed by the experiment, and the mode of cleavage.

In order to obtain binding curves and equilibrium constants for an intermolecular interaction, a series of serial dilutions of the binding molecule (e.g., a nucleic acid substrate or an RNA binding protein) can be analyzed. Preferably, the ligand concentrations should span a range from 0% to >99% saturation of all binding sites. This requires a concentration range of four orders of magnitude for even a single binding site. Site heterogeneity can increase the optimal range. The ligand concentrations in the binding reaction mixtures should define an evenly spaced, logarithmic series with at least several points to define each asymptote of the titration curve (see, e.g., Ausubel, supra).

The RNA footprinting reaction involves treating the RNA molecule in a manner such that the RNA is partially cleaved, where cleavage is restricted to solvent accessible regions. Preferably, the cleavage reaction does not discriminate in its specificity, i.e., all linkages are cleaved regardless of the identity of the linked bases, in a sequence-independent manner. In one aspect, the RNA molecule is partially digested by means of a nuclease, preferably an RNase or plurality of RNases. Examples of nucleases that might be used include RNase U2, RNase T1 and Rnase T2 (Donis-Keller et al. (1987) Nucleic Acids Res. 4:2527-38; Boguski et al. (1980) J. of Biol. Chem. 265:2160-63).

Alternatively, the digestion can be accomplished chemically using, e.g., hydroxyl radicals generated by Fe(EDTA)²⁻(Tullius and Dombroski (1986) Proc. Natl. Acad. Sci. USA 83:5469; Hampel et al. (1998) Biochemistry 37:14672-82; Latham & Cech (1989) Science 245:276-82; Cleander & Cech (1991) Science 251:401-407), Methidiumpropyl-EDTA●Fe(II)(Van Dyke and Dervan (1983) Nuc. Acids Res. 11:5555) or Cu(phen)2+(Spassky and Sigman (1985) Biochemistry 24:8050). In a particularly preferred embodiment of the invention, hydroxyl radicals generated by Fe(EDTA)²⁻are used to cleave unprotected RNA. The chemistry of hydroxyl radical induced polynucleotide cleavage is described, for example, in Balasubramanian et al., (1998) Proc. Natl. Acad. Sci. USA 95:9738-43, incorporated by reference herein in its entirety. A non-limiting example of how the footprinting reaction can be accomplished using hydroxyl radical is provided in the Examples infra.

Normally an RNA footprinting experiment of the instant invention will be carried out under two or more different conditions for comparison of relative solvent accessibility. For example, if the RNA molecule is a ribozyme, footprinting reactions of free ribozyme and ribozyme in the presence of substrate can be compared to elucidate the site of substrate interaction. An example of this type of use of the invention is provided in the Examples. The experiment can be undertaken using varying amounts of ribozyme and/or substrate to determine the thermodynamics or kinetics of substrate binding. Alternatively, the experiment can involve comparing free RNA with RNA in the presence of an RNA binding protein. In another embodiment of the invention, the method can be used to probe RNA structure under varying conditions that might influence the RNA molecules secondary structure, e.g, different temperatures, pH, ion concentrations, etc.

An important element of the instant invention that distinguishes it over previously available RNA footprinting protocols is the use of high performance liquid chromatography (HPLC) rather than electrophoresis to effect detection and quantification of the RNA cleavage products. The use of HPLC instead of electrophoresis results in a number of advantages, including shorter analysis times, more reproducible data, convenience, ease of use, and improved capability for high-throughput and automation.

As with gel electrophoresis, HPLC is used to separate out RNA cleavage products on the basis of size. In the absence of an interaction rendering a phosphodiester bond inaccessible to solvent, cleavage at each phosphodiester bond will result in a chromatographic peak corresponding to each nucleotide in the sequence being analyzed. Assuming that chromatography is performed under denaturing conditions and that a single end (typically the 5′ end) of the RNA moleucle is labeled, each labeled chromatographic peak will share a common, labeled 5′-end, and the length (and hence the location of the peak in the chromatogram) will depend upon the 3′ end (i.e., the site of cleavage). FIG. 1 depicts a such a chromatogram, representing the substrate strand of hairpin ribozyme that has undergone alkali hydrolysis. FIG. 2 shows a comparison of the footprinting chromatograms obtained for the substrate strand in free solution and in complex with ribozyme that has undergone cleavage by hydroxyl radicals. Reduced relative peak size indicates bases where ribozyme complex-interactions protect against cleavage.

In order to assign the chromatographic peaks to the corresponding site of cleavage, i.e, the ′-end of the fragment, it is normally advisable to run a parallel reaction in order to “phase” the footprinting chromatogram. For each reaction, one strand is typically labeled at the 5′-end, so that detected peaks share a common 5′-end. Thus, the length of each RNA fragment is a function of location of the 3′-end, which depends upon where the original RNA strand was cleaved. Fragments sharing a common 3′-end will elute from the column at the same time, assuming that the chromatographic conditions remain relatively constant between the two run (such reproducibility can be achieved using the preferred modes of HPLC described infra, e.g., MIPC). Thus, the 3′ end of a peak can be determined if the identity of a co-eluting peak generated in the phasing reaction is known.

In a preferred embodiment of the invention, the phasing reaction is an RNA sequencing reaction, i.e., a reaction that cleaves RNA only after a specific base (or some subset of the four bases that make up RNA). For example, Hampel et al., supra, describe phasing (in the context of gel electrophoresis) a RNA footprinting reaction by reference to a partial ribonuclease T1 digest and alkali hydrolysis ladders. Other suitable RNase sequencing reactions are Donis-Keller et al. and Boguski et al., supra. Thus, in a preferred embodiment of the invention, the RNA being analyzed is subjected in parallel to an RNA sequencing reaction and used to generate an HPLC chromatogram for phasing the RNA footprinting chromatogram.

An important element of the instant invention that makes it superior to previously available sequencing ladder is the use of high performance liquid chromatography (HPLC) rather than electrophoresis to separate and detect the RNA fragments. The use of HPLC instead of electrophoresis results in a number of advantages, including shorter analysis times, more reproducible data, convenience, ease of use, improved capability for high-throughput and automation, enhanced ability to resolve and detect very small RNA fragments.

In preferred embodiment of the invention ion pairing reverse phase HPLC (IP-RP-HPLC) is used to analyze the RNA cleavage products. IP-RP-HPLC is a form of chromatography particularly suited to the analysis of both single and double stranded polynucleotides, and is characterized by the use of a reversed phase (i.e., hydrophobic) stationary phase and a mobile phase that includes an alkylated cation (e.g., triethylammonium) that is believed to form a bridging interaction between the negatively charged polynucleotide and non-polar stationary phase. The alkylated cation-mediated interaction of RNA and stationary phase can be modulated by the polarity of the mobile phase, conveniently adjusted by means of a solvent that is less polar than water, e.g., acetonitrile. Performance is enhanced by the use of a non-porous separation medium, as described in U.S. Pat. No. 5,585,236. The most preferred method of analysis by means of Matched Ion Polynucleotide Chromatography (MIPC), a superior form of IP-RP-HPLC described in U.S. Pat. Nos. 5,585,236, 6,066,258 and 6,056,877 and PCT Publication Ser. Nos. WO98/48913, WO98/48914, WO98/56797, WO98/56798, incorporated herein by reference in their entirety. MIPC is characterized by the use of solvents and chromatographic surfaces that are substantially free of multivalent cation contamination that can interfere with polynucleotide separation. In the practice of the invention, a preferred system for performing MIPC separations is that provided by Transgenomic, Inc. (San Jose, Calif.) under the trademark WAVE®.

Separation by RP-IP-HPLC, including MIPC, occurs at the non-polar surface of a separation medium. In one embodiment, the non-polar surfaces comprise the surfaces of polymeric beads. In an alternative embodiment, the surfaces comprise the surfaces of interstitial spaces in a molded polymeric monolith, described in more detail infra. For purposes of simplifying the description of the invention and not by way of limitation, the separation of polynucleotides using nonporous beads, and the preparation of such beads, will be primarily described herein, it being understood that other separation surfaces, such as the interstitial surfaces of polymeric monoliths, are intended to be included within the scope of this invention.

In general, in order to be suitable for use in IP-RP-HPLC a separation medium should have a surface that is either intrinsically non-polar or bonded with a material that forms a surface having sufficient non-polarity to interact with a counterion agent.

In one aspect of the invention, IP-RP-HPLC detection is accomplished using a column filled with nonporous polymeric beads having an average diameter of about 0.5-100 microns; preferably, 1-10 microns; more preferably, 1-5 microns. Beads having an average diameter of 1.0-3.0 microns are most preferred.

In a preferred embodiment of the invention, the chromatographic separation medium comprises nonporous beads, i.e., beads having a pore size that essentially excludes the polynucleotides being separated from entering the bead, although porous beads can also be used. As used herein, the term “nonporous” is defined to denote a bead that has surface pores having a diameter that is sufficiently small so as to effectively exclude the smallest RNA fragment in the separation in the solvent medium used therein. Included in this definition are polymer beads having these specified maximum size restrictions in their natural state or which have been treated to reduce their pore size to meet the maximum effective pore size required.

The surface conformations of nonporous beads of the present invention can include depressions and shallow pit-like structures that do not interfere with the separation process. A pretreatment of a porous bead to render it nonporous can be effected with any material which will fill the pores in the bead structure and which does not significantly interfere with the MIPC process.

Pores are open structures through which mobile phase and other materials can enter the bead structure. Pores are often interconnected so that fluid entering one pore can exit from another pore. Without intending to be bound by any particular theory, it is believed that pores having dimensions that allow movement of the polynucleotide into the interconnected pore structure and into the bead impair the resolution of separations or result in separations that have very long retention times.

Non-porous polymeric beads useful in the practice of the present invention can be prepared by a two-step process in which small seed beads are initially produced by emulsion polymerization of suitable polymerizable monomers. The emulsion polymerization procedure is a modification of the procedure of Goodwin, et al. (Colloid & Polymer Sci., 252:464-471 (1974)). Monomers that can be used in the emulsion polymerization process to produce the seed beads include styrene, alkyl substituted styrenes, alpha-methyl styrene, and alkyl substituted alpha-methyl styrene. The seed beads are then enlarged and, optionally, modified by substitution with various groups to produce the nonporous polymeric beads of the present invention.

The seed beads produced by emulsion polymerization can be enlarged by any known process for increasing the size of the polymer beads. For example, polymer beads can be enlarged by the activated swelling process disclosed in U.S. Pat. No. 4,563,510. The enlarged or swollen polymer beads are further swollen with a crosslinking polymerizable monomer and a polymerization initiator. Polymerization increases the crosslinking density of the enlarged polymeric bead and reduces the surface porosity of the bead. Suitable crosslinking monomers contain at least two carbon-carbon double bonds capable of polymerization in the presence of an initiator. Preferred crosslinking monomers are divinyl monomers, preferably alkyl and aryl (phenyl, naphthyl, etc.) divinyl monomers and include divinyl benzene, butadiene, etc. Activated swelling of the polymeric seed beads is useful to produce polymer beads having an average diameter ranging from 1 up to about 100 microns.

Alternatively, the polymer seed beads can be enlarged simply by heating the seed latex resulting from emulsion polymerization. This alternative eliminates the need for activated swelling of the seed beads with an activating solvent. Instead, the seed latex is mixed with the crosslinking monomer and polymerization initiator described above, together with or without a water-miscible solvent for the crosslinking monomer. Suitable solvents include acetone, tetrahydrofuran (THF), methanol, and dioxane. The resulting mixture is heated for about 1-12 hours, preferably about 4-8 hours, at a temperature below the initiation temperature of the polymerization initiator, generally, about 10° C.-80° C., preferably 30° C.-60° C. Optionally, the temperature of the mixture can be increased by 10-20% and the mixture heated for an additional 1 to 4 hours. The ratio of monomer to polymerization initiator is at least 100:1, preferably in the range of about 100:1 to about 500:1, more preferably about 200:1 in order to ensure a degree of polymerization of at least 200. Beads having this degree of polymerization are sufficiently pressure-stable to be used in HPLC applications. This thermal swelling process allows one to increase the size of the bead by about 110-160% to obtain polymer beads having an average diameter up to about 5 microns, preferably about 2-3 microns. The thermal swelling procedure can, therefore, be used to produce smaller particle sizes previously accessible only by the activated swelling procedure.

Following thermal enlargement, excess crosslinking monomer is removed and the particles are polymerized by exposure to ultraviolet light or heat. Polymerization can be conducted, for example, by heating of the enlarged particles to the activation temperature of the polymerization initiator and continuing polymerization until the desired degree of polymerization has been achieved. Continued heating and polymerization allows one to obtain beads having a degree of polymerization greater than 500.

For use in the present invention, packing material disclosed by U.S. Pat. No. 4,563,510 can be modified through substitution of the polymeric beads with alkyl groups or can be used in its unmodified state. For example, the polymer beads can be alkylated with 1 or 2 carbon atoms by contacting the beads with an alkylating agent, such as methyl iodide or ethyl iodide. Alkylation can be achieved by mixing the polymer beads with the alkyl halide in the presence of a Friedel-Crafts catalyst to effect electrophilic aromatic substitution on the aromatic rings at the surface of the polymer blend. Suitable Friedel-Crafts catalysts are well-known in the art and include Lewis acids such as aluminum chloride, boron trifluoride, tin tetrachloride, etc. The beads can be hydrocarbon substituted by substituting the corresponding hydrocarbon halide for methyl iodide in the above procedure, for example.

The term alkyl as used herein in reference to the beads useful in the practice of the present invention is defined to include alkyl and alkyl substituted aryl groups, having from 1 to 1,000,000 carbons, the alkyl groups including straight chained, branch chained, cyclic, saturated, unsaturated nonionic functional groups of various types including aldehyde, ketone, ester, ether, alkyl groups, and the like, and the aryl groups including as monocyclic, bicyclic, and tricyclic aromatic hydrocarbon groups including phenyl, naphthyl, and the like. Methods for alkyl substitution are conventional and well-known in the art and are not an aspect of this invention. The substitution can also contain hydroxy, cyano, nitro groups, or the like which are considered to be non-polar, reverse phase functional groups.

Non-limiting examples of base polymers suitable for use in producing such polymer beads include mono- and di-vinyl substituted aromatics such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(styrene-divinylbenzene) and poly(ethylvinylbenzene-divinylbenzene). Methods for making beads from these polymers are conventional and well known in the art (for example, see U.S. Pat. No. 4,906,378). The physical properties of the surface and near-surface areas of the beads are the primary determinant of chromatographic efficiency. The polymer, whether derivatized or not, should provide a nonporous, non-reactive, and non-polar surface for the MICP separation. In a particularly preferred embodiment of the invention, the separation medium consists of octadecyl modified, nonporous alkylated poly(styrene-divinylbenzene) beads. Separation columns employing these particularly preferred beads, referred to as DNASepe® columns, are commercially available from Transgenomic, Inc.

A separation bead used in the invention can comprise a nonporous particle which has non-polar molecules or a non-polar polymer attached to or coated on its surface. In general, such beads comprise nonporous particles which have been coated with a polymer or which have substantially all surface substrate groups reacted with a non-polar hydrocarbon or substituted hydrocarbon group, and any remaining surface substrate groups endcapped with a tri(lower alkyl)chlorosilane or tetra(lower alkyl)dichlorodisilazane as described in U.S Pat. No. 6,056,877.

The nonporous particle is preferably an inorganic particle, but can be a nonporous organic particle. The nonporous particle can be, for example, silica, silica carbide, silica nitrite, titanium oxide, aluminum oxide, zirconium oxide, carbon, insoluble polysaccharides such as cellulose, or diatomaceous earth, or any of these materials which have been modified to be nonporous. Examples of carbon particles include diamond and graphite which have been treated to remove any interfering contaminants. The preferred particles are essentially non-deformable and can withstand high pressures. The nonporous particle is prepared by known procedures. The preferred particle size is about 0.5-100 microns; preferably, 1-10 microns; more preferably, 1-5 microns. Beads having an average diameter of 1.0-3.0 microns are most preferred.

Because the chemistry of preparing conventional silica-based reverse phase HPLC materials is well-known, most of the description of non-porous beads suitable for use in the instant invention is presented in reference to silica. It is to be understood, however, that other nonporous particles, such as those listed above, can be modified in the same manner and substituted for silica. For a description of the general chemistry of silica, see Poole, Colin F. and Salwa K. Poole, Chromatography Today, Elsevier:New York (1991), pp. 313-342 and Snyder, R. L. and J. J. Kirkland, Introduction to Modern Liquid Chromatography, 2² nd ed., John Wiley & Sons, lnc.:New York (1979), pp.272-278, the disclosures of which are hereby incorporated herein by reference in their entireties.

The nonporous beads of the invention are characterized by having minimum exposed silanol groups after reaction with the coating or silating reagents. Minimum silanol groups are needed to reduce the interaction of the RNA with the substrate and also to improve the stability of the material in a high pH and aqueous environment. Silanol groups can be harmful because they can repel the negative charge of the RNA molecule, preventing or limiting the interaction of the RNA with the stationary phase of the column. Another possible mechanism of interaction is that the silanol can act as ion exchange sites, taking up metals such as iron (III) or chromium (III). Iron (II) or other metals that are trapped on the column can distort the RNA peaks or even prevent RNA from being eluted from the column.

Silanol groups can be hydrolyzed by the aqueous-based mobile phase. Hydrolysis will increase the polarity and reactivity of the stationary phase by exposing more silanol sites, or by exposing metals that can be present in the silica core. Hydrolysis will be more prevalent with increased underivatized silanol groups. The effect of silanol groups on the RNA separation depends on which mechanism of interference is most prevalent. For example, iron (III) can become attached to the exposed silanol sites, depending on whether the iron (II) is present in the eluent, instrument or sample.

The effect of metals can only occur if metals are already present within the system or reagents. Metals present within the system or reagents can get trapped by ion exchange sites on the silica. However, if no metals are present within the system or reagents, then the silanol groups themselves can cause interference with RNA separations. Hydrolysis of the exposed silanol sites by the aqueous environment can expose metals that might be present in the silica core.

Fully hydrolyzed silica contains a concentration of about 8 μmoles of silanol groups per square meter of surface. At best, because of steric considerations, a maximum of about 4.5 μmoles of silanol groups per square meter can be reacted, the remainder of the silanol being sterically shielded by the reacted groups. Minimum silanol groups is defined as reaching the theoretical limit of or having sufficient shield to prevent silanol groups from interfering with the separation.

Numerous methods exist for forming nonporous silica core particles. For example, sodium silicate solution poured into methanol will produce a suspension of finely divided spherical particles of sodium silicate. These particles are neutralized by reaction with acid. In this way, globular particles of silica gel are obtained having a diameter of about 1-2 microns. Silica can be precipitated from organic liquids or from a vapor. At high temperature (about 2000° C.), silica is vaporized, and the vapors can be condensed to form finely divided silica either by a reduction in temperature or by using an oxidizing gas. The synthesis and properties of silica are described by R. K. IIer in The Chemistry of Silica, Solubility, Polymerization, Colloid and Surface Properties, and Biochemistry, John Wiley & Sons:New York (1979).

W. Stöber et al. described controlled growth of monodisperse silica spheres in the micron size range in J. Colloid and Interface Sci., 26:62-69 (1968). Stöber et al. describe a system of chemical reactions which permit the controlled growth of spherical silica particles of uniform size by means of hydrolysis of alkyl silicates and subsequent condensation of silicic acid in alcoholic solutions. Ammonia is used as a morphological catalyst. Particle sizes obtained in suspension range from less than 0.05 μm to 2 μm in diameter.

To prepare a nonporous bead, the nonporous particle can be coated with a polymer or reacted and endcapped so that substantially all surface substrate groups of the nonporous particle are blocked with a non-polar hydrocarbon or substituted hydrocarbon group. This can be accomplished by any of several methods described in U.S. Pat. No. 6,056,877. Care should be taken during the preparation of the beads to ensure that the surface of the beads has minimum silanol or metal oxide exposure and that the surface remains nonporous. Nonporous silica core beads can be obtained from Micra Scientific (Northbrook, Ill.) and from Chemie Uetikkon (Lausanne, Switzerland).

In another embodiment of the present invention, the IP-RP-HPLC separation medium can be in the form of a polymeric monolith, e.g., a rod-like monolithic column. A monolith is a polymer separation media, formed inside a column, having a unitary structure with through pores or interstitial spaces that allow eluting solvent and analyte to pass through and which provide the non-polar separation surface, as described in U.S. Pat. No. 6,066,258 and U.S. patent application Ser. No. 09/562,069. The interstitial separation surfaces can be porous, but are preferably nonporous. The separation principles involved parallel those encountered with bead-packed columns. As with beads, pores traversing the monolith must be compatible with and permeable to RNA. In a preferred embodiment, the rod is substantially free of contamination capable of reacting with RNA and interfering with its separation, e.g., multivalent cations.

A molded polymeric monolith rod that can be used in practicing the present invention can be prepared, for example, by bulk free radical polymerization within the confines of a chromatographic column. The base polymer of the rod can be produced from a variety of polymerizable monomers. For example, the monolithic rod can be made from polymers, including mono- and di-vinyl substituted aromatic compounds such as styrene, substituted styrenes, alpha-substituted styrenes and divinylbenzene; acrylates and methacrylates; polyolefins such as polypropylene and polyethylene; polyesters; polyurethanes; polyamides; polycarbonates; and substituted polymers including fluorosubstituted ethylenes commonly known under the trademark TEFLON. The base polymer can also be mixtures of polymers, non-limiting examples of which include poly(glycidyl methacrylate-co-ethylene dimethacrylate), poly(styrene-divinylbenzene) and poly(ethylvinylbenzene-divinylbenzene. The rod can be unsubsituted or substituted with a substituent such as a hydrocarbon alkyl or an aryl group. The alkyl group optionally has 1 to 1,000,000 carbons inclusive in a straight or branched chain, and includes straight chained, branch chained, cyclic, saturated, unsaturated nonionic functional groups of various types including aldehyde, ketone, ester, ether, alkyl groups, and the like, and the aryl groups includes as monocyclic, bicyclic, and tricyclic aromatic hydrocarbon groups including phenyl, naphthyl, and the like. In a preferred embodiment, the alkyl group has 1-24 carbons. In a more preferred embodiment, the alkyl group has 1-8 carbons. The substitution can also contain hydroxy, cyano, nitro groups, or the like which are considered to be non-polar, reverse phase functional groups. Methods for hydrocarbon substitution are conventional and well-known in the art and are not an aspect of this invention. The preparation of polymeric monoliths is by conventional methods well known in the art as described in the following references: Wang et al.(1994) J. Chromatog. A 699:230; Petro et al. (1996) Anal. Chem. 68:315 and U.S. Pat. Nos. 5,334,310; 5,453,185 and 5,522,994. Monolith or rod columns are commercially available form Merck & Co (Darmstadt, Germany).

The separation medium can take the form of a continuous monolithic silica gel. A molded monolith can be prepared by polymerization within the confines of a chromatographic column (e.g., to form a rod) or other containment system. A monolith is preferably obtained by the hydrolysis and polycondensation of alkoxysilanes. A preferred monolith is derivatized in order to produce non-polar interstitial surfaces. Chemical modification of silica monoliths with ocatdecyl, methyl or other ligands can be carried out. An example of a preferred derivatized monolith is one that is polyfunctionally derivatized with octadecylsilyl groups. The preparation of derivatized silica monoliths can be accomplished using conventional methods well known in the art as described in the following references which are hereby incorporated in their entirety herein: U.S Pat. No. 6,056,877, Nakanishi, et al., J. Sol-Gel Sci. Technol. 8:547 (1997); Nakanishi, et al., Bull, Chem. Soc. Jpn. 67:1327 (1994); Cabrera, et al., Trends Analytical Chem. 17:50 (1998); Jinno, et al., Chromatographia 27:288 (1989).

MIPC is characterized by the use of a separation medium having low amounts of metal contaminants or other contaminants that can bind RNA. Preferred beads and monoliths have been produced under conditions where precautions have been taken to substantially eliminate any multivalent cation contaminants (e.g. Fe(III), Cr(III), or colloidal metal contaminants), including a decontamination treatment, e.g., an acid wash treatment. Only very pure, non-metal containing materials should be used in the production of the beads in order to minimize the metal content of the resulting beads.

In addition to the separation medium being substantially metal-free, to achieve optimum peak separation the separation column and all process solutions held within the column or flowing through the column are preferably substantially free of multivalent cation contaminants (e.g. Fe(III), Cr(III), and colloidal metal contaminants). As described in U.S. Pat. Nos. 5,772,889, 5,997,742 and 6,017,457, this can be achieved by supplying and feeding solutions that enter the separation column with components that have process solution-contacting surfaces made of material that does not release multivalent cations into the process solutions held within or flowing through the column, in order to protect the column from multivalent cation contamination. The process solution-contacting surfaces of the system components are preferably material selected from the group consisting of titanium, coated stainless steel, passivated stainless steel, and organic polymer. Metals found in stainless steel, for example, do not harm the separation, unless they are in an oxidized or colloidal partially oxidized state. For example, 316 stainless steel frits are acceptable in column hardware, but surface oxidized stainless steel frits harm the RNA separation.

For additional protection, multivalent cations in mobile phase solutions and sample solutions entering the column can be removed by contacting these solutions with multivalent cation capture resin before the solutions enter the column to protect the separation medium from multivalent cation contamination. The multivalent capture resin is preferably cation exchange resin and/or chelating resin.

Trace levels of multivalent cations anywhere in the solvent flow path can cause a significant deterioration in the resolution of the separation after multiple uses of an IP-RP-HPLC column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the MIPC column. These, and similar measures, taken to prevent system contamination with multivalent cations have resulted in extended column life and reduced analysis downtime.

There are two places where multivalent-cation-binding agents, e.g., chelators, are used in MIPC separations. In one embodiment, these binding agents can be incorporated into a solid through which the mobile phase passes. Contaminants are trapped before they reach places within the system that can harm the separation. In these cases, the functional group is attached to a solid matrix or resin (e.g., a flow-through cartridge, usually an organic polymer, but sometimes silica or other material). The capacity of the matrix is preferably about 2 mequiv./g. An example of a suitable chelating resin is available under the trademark CHELEX 100 (Dow Chemical Co.) containing an iminodiacetate functional group.

In another embodiment, the multivalent cation-binding agent can be added to the mobile phase. The binding functional group is incorporated into an organic chemical structure. The preferred multivalent cation-binding agent fulfills three requirements. First, it is soluble in the mobile phase. Second, the complex with the metal is soluble in the mobile phase. Multivalent cation- binding agents such as EDTA fulfill this requirement because both the chelator and the multivalent cation-binding agent-metal complex contain charges, which makes them both water-soluble. Also, neither precipitate when acetonitrile, for example, is added. The solubility in aqueous mobile phase can be enhanced by attaching covalently bound ionic functionality, such as, sulfate, carboxylate, or hydroxy. A preferred multivalent cation-binding agent can be easily removed from the column by washing with water, organic solvent or mobile phase. Third, the binding agent must not interfere with the chromatographic process.

The multivalent cation-binding agent can be a coordination compound. Examples of preferred coordination compounds include water soluble chelating agents and crown ethers. Non-limiting examples of multivalent cation-binding agents which can be used in the present invention include acetylacetone, alizarin, aluminon, chloranilic acid, kojic acid, morin, rhodizonic acid, thionalide, thiourea, α-furildioxime, nioxime, salicylaldoxime, dimethylglyoxime, α-furildioxime, cupferron, α-nitroso-β-naphthol, nitroso-R-salt, diphenylthiocarbazone, diphenylcarbazone, eriochrome black T, PAN, SPADNS, glyoxal-bis(2-hydroxyanil), murexide, α-benzoinoxime, mandelic acid, anthranilic acid, ethylenediamine, glycine, triaminotriethylamine, thionalide, triethylenetetramine, EDTA, metalphthalein, arsonic acids, α,α′-bipyridine, 4-hydroxybenzothiazole, 8-hydroxyquinaldine, 8-hydroxyquinoline, 1,10-phenanthroline, picolinic acid, quinaldic acid, α,α′,α″-terpyridyl, 9-methyl-2,3,7-trihydroxy-6-fluorone, pyrocatechol, salicylic acid, tiron, 4-chloro-1,2-dimercaptobenzene, dithiol, mercaptobenzothiazole, rubeanic acid, oxalic acid, sodium diethyidithiocarbarbamate, and zinc dibenzyldithiocarbamate. These and other examples are described by Perrin in Organic Complexing Reagents: Structure, Behavior, and Application to Inorganic Analysis, Robert E. Krieger Publishing Co. (1964). In the present invention, a preferred multivalent cation-binding agent is EDTA.

To achieve high-resolution chromatographic separations of polynucleotides, it is generally necessary to tightly pack the chromatographic column with the solid phase polymer beads. Any known method of packing the column with a column packing material can be used in the present invention to obtain adequate high-resolution separations. Typically, a slurry of the polymer beads is prepared using a solvent having a density equal to or less than the density of the polymer beads. The column is then filled with the polymer bead slurry and vibrated or agitated to improve the packing density of the polymer beads in the column. Mechanical vibration or sonication is typically used to improve packing density.

For example, to pack a 50×4.6 mm I.D. column, 2.0 grams of beads can be suspended in 10 mL of methanol with the aid of sonication. The suspension is then packed into the column using 50 mL of methanol at 8,000 psi of pressure. This improves the density of the packed bed.

There are several types of counterions suitable for use with IP-RP-HPLC. These include a mono-, di-, or trialkylamine that can be protonated to form a positive counter charge or a quaternary alkyl substituted amine that already contains a positive counter charge. The alkyl substitutions may be uniform (for example, triethylammonium acetate or tetrapropylammonium acetate) or mixed (for example, propyldiethylammonium acetate). The size of the alkyl group may be small (methyl) or large (up to 30 carbons) especially if only one of the substituted alkyl groups is large and the others are small. For example octyldimethylammonium acetate is a suitable counterion agent. Preferred counterion agents are those containing alkyl groups from the ethyl, propyl or butyl size range.

Without intending to be bound by any particular theory, it is believed the alkyl group functions by imparting a nonpolar character to the RNA through an ion pairing process so that the RNA can interact with the nonpolar surface of the separation media. The requirements for the degree of nonpolarity of the counterion-RNA pair depends on the polarity of the separation media, the solvent conditions required for separation, the particular size and type of fragment being separated. For example, if the polarity of the separation media is increased, then the polarity of the counterion agent may have to be adjusted to match the polarity of the surface and increase interaction of the counterion-RNA pair. In general, as the size and hydrophobicity of the alkyl group is increased, the separation is less influenced by RNA sequence and base composition, but rather is based predominately on RNA sequence length.

In some cases, it may be desired to increase the range of concentration of organic solvent used to perform the separation. For example, increasing the alkyl chain length on the counterion agent will increase the nonpolarity of the counterion-RNA pair resulting in the need to either increase the concentration of the mobile phase organic solvent, or increase the strength of the organic solvent type, e.g., acetonitrile is about two times more effective than methanol for eluting RNA. There is a positive correlation between concentration of the organic solvent required to elute a fragment from the column and the length of the fragment. However, at high organic solvent concentrations, the polynucleotide can precipitate. To avoid precipitation, a more non-polar organic solvent and/or a smaller counterion alkyl group can be used. The alkyl group on the counterion reagent can also be substituted with halides, nitro groups, or the like to modulate polarity.

The mobile phase preferably contains a counterion agent. Typical counterion agents include trialkylammonium salts of organic or inorganic acids, such as lower alkyl primary, secondary, and lower tertiary amines, lower trialkyammonium salts and lower quaternary alkyalmmonium salts. Lower alkyl refers to an alkyl radical of one to six carbon atoms, as exemplified by methyl, ethyl, n-butyl, i-butyl, t-butyl, isoamyl, n-pentyl, and isopentyl. Examples of counterion agents include octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, and tetrabutylammonium acetate. Although the anion in the above examples is acetate, other anions may also be used, including carbonate, phosphate, sulfate, nitrate, propionate, formate, chloride, and bromide, or any combination of cation and anion. These and other agents are described by Gjerde, et al. in Ion Chromatography, 2nd Ed., Dr. Alfred Hüthig Verlag Heidelberg (1987). In a particularly preferred embodiment of the invention the counterion is tetrabutylammonium bromide (TBAB) is preferred, although other quaternary ammonium reagents such as tetrapropyl or tetrabutyl ammonium salts can be used. Alternatively, a trialkylammonium salt, e.g., triethylammonium acetate (TEAA) can be used.

The pH of the mobile phase is preferably within the range of about pH 5 to about pH 9, and optimally within the range of about pH 6 to about pH 7.5.

In a preferred embodiment of the method, optimum peak resolution the is achieved by carrying out the separation under conditions effective to denature the secondary structure of the RNA molecules. The temperature required to achieve denaturation will vary, depending upon the nature of the column, the mobile phase and counterion agent used, and the melting properties of the RNA being separated. For example, the denaturation can be accomplished by conducting the elution at a temperature greater than about 6° C. and more preferably above 70° C. An operable temperature is within the range of about 40° C. to about 80° C. In a particularly preferred embodiment of the invention, where the separation medium is octadecyl modified, nonporous alkylated poly(styrenedivinylbenzene) beads, the aqueous mobile phase contains acetonitrile and TBAB is used as a counterion, the column temperature is preferably greater than 50° C., more preferably between about 50° C. and 80° C., and most preferably about 70° C. Suitable conditions for separating RNA by IP-RP-HPLC are described in U.S. patent application Ser. No. 09/557,424, incorporated by reference herein in its entirety.

The temperature at which the separation is performed affects the choice of organic solvents used in the separation, and vice versa. The solvent affects the temperature at which an RNA molecule denatures. Furthermore, the polarity of a solvent affects the distribution of the RNA between the mobile phase and the stationary phase.

An organic solvent that is water-soluble is preferably used, e.g., an alcohol, nitrile, dimethylformamide (DMF), tetrahydrofuran (THF), ester, or ether. Water-soluble solvents are defined as those that exist as a single phase with aqueous systems under all conditions of operation of the present invention. For example, acetonitrile and 1-propanol have polarity and solubility properties that are particularly suited for use in the present invention. However, methanol can be a good alternative that reduces cost and toxicity concerns. Solvents that are particularly preferred for use in the method of this invention include methanol, ethanol, 2-propanol, 1-propanol, tetrahydrofuran (THF), and acetonitrile, with acetonitrile being most preferred overall.

In performing IP-RP-HPLC and MIPC, even trace levels of multivalent cations anywhere in the solvent flow path can cause a significant deterioration in the resolution of the separation after multiple uses of a column. This can result in increased cost caused by the need to purchase replacement columns and increased downtime. Therefore, effective measures are preferably taken to prevent multivalent metal cation contamination of the separation system components, including separation media and mobile phase contacting. These measures include, but are not limited to, washing protocols to remove traces of multivalent cations from the separation media and installation of guard cartridges containing cation capture resins, in line between the mobile phase reservoir and the column. These, and similar measures, taken to prevent system contamination with multivalent cations have resulted in extended column life and reduced analysis downtime.

In some instances, in order to optimize column life and maintain effective separation performance, it will be desirable to periodically run an aqueous solution of multivalent cation-binding agent through the column, e.g., after about 500 uses or when the performance starts to degrade. Examples of suitable cation-binding agents are as described hereinabove.

The concentration of a solution of the cation-binding agent can be between 0.01 M and 1 M. In a preferred embodiment, the column washing solution contains EDTA at a concentration of about 0.03 to 0.1 M.

In another embodiment, the solution contains an organic solvent selected from the group consisting of acetonitrile, ethanol, methanol, 2-propanol, and ethyl acetate. A preferred solution contains at least 2% organic solvent to prevent microbial growth. In a most preferred embodiment a solution containing 25% acetonitrile is used to wash a column. The multivalent cation-binding solution can contain a counterion agent as described hereinabove.

In one embodiment of a column washing procedure, the separation column is washed with the multivalent cation-binding solution at an elevated temperature in the range of 50° C. to 80° C. In a preferred embodiment the column is washed with a solution containing EDTA, TEAA, and acetonitrile, in the 70° C. to 80° C. temperature range. In a specific embodiment, the solution contains 0.032 M EDTA, 0.1 M TEAA, and 25% acetonitrile.

Column washing can range from 30 seconds to one hour. In a preferred procedure, the column is washed with multivalent cation-binding agent for 30 to 60 minutes at a flow rate preferably in the range of about 0.05 to 1.0 mL/min.

Other treatments for washing a column can also be used alone or in combination with those indicated hereinabove. These include: use of high pH washing solutions (e.g., pH 10-12), use of denaturants such as urea or formamide, and reverse flushing the column with washing solution.

MIPC separation efficiency can be preserved by storing the column separation media in the presence of a solution of multivalent cation-binding agent. The solution of binding agent may also contain a counterion agent. Any of the multivalent cation-binding agents, counterion agents, and solvents described hereinabove are suitable for the purpose of storing a MIPC column. In a preferred embodiment, a column packed with MIPC separation media is stored in an organic solvent containing a multivalent cationbinding agent and a counterion agent. An example of this preferred embodiment is 0.032 M EDTA and 0.1 M TEAA in 25% aqueous acetonitrile. In preparation for storage, a solution of multivalent cation-binding agent, as described above, is passed through the column for about 30 minutes. The column is then disconnected from the HPLC apparatus and the column ends are capped with commercially available threaded end caps made of material which does not release multivalent cations. Such end caps can be made of coated stainless steel, titanium, organic polymer or any combination thereof.

High pressure pumps are used for pumping mobile phase in the systems described in U.S. Pat. No. 5,585,236 to Bonn and in U.S. Pat. No. 5,772,889 to Gjerde. It will be appreciated that other methods are known for driving mobile phase through separation media and can be used in carrying out the analysis described in the present invention. A non-limiting example of such an alternative method includes “capillary electrochromatography” (CEC) in which an electric field is applied across capillary columns packed with microparticles and the resulting electroosmotic flow acts as a pump for chromatography. Electroosmosis is the flow of liquid, in contact with a solid surface, under the influence of a tangentially applied electric field. The technique combines the advantages of the high efficiency obtained with capillary electrophoretic separations, such as capillary zone electrophoresis, and the general applicability of HPLC. CEC has the capability to drive the mobile phase through columns packed with chromatographic particles, especially small particles, when using electroosmotic flow. High efficiencies can be obtained as a result of the plug-like flow profile. In the use of CEC in the present invention, solvent gradients are used and rapid separations can be obtained using high electric fields. The following references describing CEC are each incorporated in their entirety herein: Dadoo, et al, LC-GC 15:630 (1997); Jorgenson, et al., J. Chromatog. 218:209 (1981); Pretorius, et al., J. Chromatog. 99:23 (1974); and the following U.S. Pat. Nos. to Dadoo 5,378,334 (1995), 5,342,492 (1994), and 5,310,463 (1994). In the operation of this aspect of the present invention, the capillaries are packed, either electrokinetically or using a pump, with the separation beads described in the present specification. In another embodiment, a polymeric rod is prepared by bulk free radical polymerization within the confines of a capillary column. Capillaries are preferably formed from fused silica tubing or etched into a block. The packed capillary (e.g., a 150-μm i.d. with a 20-cm packed length and a window located immediately before the outlet frit) is fitted with frits at the inlet and outlet ends. An electric field, e.g., 2800 V/cm, is applied. Detection can be by UV absorbance or by fluorescence. A gradient of organic solvent, e.g., acetonitrile, is applied in a mobile phase containing counterion agent (e.g. 0.1 M TEAA). to elute the polynucleotides. The column temperature is maintained by conventional temperature control means. In the preferred embodiment, all of the precautions for minimizing trace metal contaminants as described hereinabove are employed in using CEC.

Other features of the invention will become apparent in the course of the following descriptions of exemplary embodiments, which are given for illustration of the invention and are not intended to be limiting thereof.

Procedures described in the past tense in the examples below have been carried out in the laboratory. Procedures described in the present tense have not yet been carried out in the laboratory, and are constructively reduced to practice with the filing of this application.

### EXAMPLE 1

### Alkali Catalyzed Hydrolysis

The oligonucleotides used in this and subsequent examples were synthesized on an Applied Biosystems 394 DNA synthesiser using cyanoethyl phosphoramidite chemistry. Following deprotection, the oligonucleotides were purified using denaturing PAGE, evaporated to dryness and desalted using a Pharmacia NAP 10 column according to the manufacturer's instructions.

20 pmoles of RNA (5′- FAM-UCGCAGUCCUAUU-3′; SEQ ID NO: 1) was added to 0.1 M NaH(CO)₃, pH 8.4 in a total volume of 20 μl. The reaction mixture was then maintained at 95° C. for 20 mins. A 5 μl sample was then analyzed by IP-RP-HPLC under denaturing conditions using an analytical size (inner dimensions 50×4.6 mm) DNASep™ column (Trangenomic, Inc) and a WAVE nucleic acid analysis system (Transgenomic, Inc.). The stationary phase of the DNASep™ column comprises octadecyl modified, nonporous poly(styrene-divinylbenzene) beads, as described in U.S. Pat. No. 6,066,258. The chromatographic separation was controlled by a WAVE® fragment analysis system (Transgenomic, Inc.; San Jose, Calif.) at 70° C. using fluorescence detection at the appropriate excitation and emission wavelengths (for FAM EX=494 and Em=525). The following elution conditions were used: Buffer A 0.0025 M Tetrabutylammonium bromide (Fluka HPLC), 1 mM EDTA (Sigma), 0.1% acetonitrile, buffer B 0.0025 M, Tetrabutylammonium bromide, 1 mM EDTA (Sigma), 70% acetonitrile starting at 25% buffer B. The gradient was extended to 35% buffer B over 1 minute at a flow rate of 0.9 ml/min, followed by an extension to 50% buffer B over 18 minutes at a flow rate of 0.9 ml/min, followed by an extension to 60% buffer B over 30 minutes at a flow rate of 0.9 ml/min.

FIG. 1 depicts the resulting chromatogram. The A-1 position contains a 2′-O methyl group that is resistant to base cleavage. This results in a block to cleavage product at this position.

### EXAMPLE 2

### Hydroxyl Radical Cleavage

RNA footprinting reactions were performed according to the method described by Hampel et al (1998) Biochemistry 37: 14672-82.

To analyze the RNA substrate strand in free solution 20 pmoles of the strand (5′-FAM-UCGCAGUCCUAUU; SEQ ID NO: 1) was added to a solution containing 50 mM NaCI, 0.1 mM Tris pH 7.4, 1mM Co²⁺(NH₃)₆ in a final volume of 15 μl. 5 μl of 100 mM Ascorbate (Aldrich), followed by 5 μl of 1.2% H₂O₂ (Aldrich), 10 μl of 0.4 mM Fe²⁺/0.8 nM EDTA (Aldrich) solution was added and rapidly mixed and incubated at room temp for 4 minutes. The reaction was then stopped by the addition of 10 μl of0.1 M thiourea (Sigma), 0.1 M EDTA solution.

Prior to IP-RP-HPLC, the reaction product was purified using a spin-column containing octadecyl modified, nonporous alkylated poly(styrene-divinylbenzene) beads, as described in U.S. application Ser. No. 09/318,407 and PCT/US00/14956. The spin columns were first incubated with 500 μl of 0.0025 M tBuBr (tetrabutylammonium bromide). A volume of 0.0025 M tBuBr equal to the reaction volume was added to the reaction mixtures and then loaded onto the column. The columns were then washed twice with 0.0025 M tBuBr containing 2 mM EDTA (pH 8.0). The RNA fragments were then eluted using 70% acetonitrile, loaded onto the DNASep™ column, and analyzed as described in Example 1.

To analyze the substrate strand in the ribozyme complex, 20 pmoles of the RNA substrate strand (5′- FAM UCGCAGUCCUAUU; SEQ ID NO: 1)), 40 pmoles of strand A (see FIG. 2 5′-GGCGUGGUACAUUACCUGGUA; SEQ ID NO: 2), 40 pmoles strand B (5′-AAAUAGAGMGCGMCCAGAGAAACACACGCC; SEQ ID NO: 3) were added to a solution containing 50 mM NaCl, 0.1 mM Tris pH 7.4, 1 mM Co²⁺(NH₃)₆ in a final volume of 15 μl. 5 μl of 100 mM Ascorbate (Aldrich), followed by 5 μl of 1.2% H₂O₂ (Aldrich), 10 μl of 0.4 Fe 2+/ 0.8 mM EDTA (Aldrich) solution was added and rapidly mixed and incubated at room temp for 4 minutes. The reaction was then stopped by the addition of 10 μl of 0.1 M thiourea (Sigma), 0.1 M EDTA . The sample was then purified using the spin columns and analyzed on the DNASep™ column as described above.

FIG. 2 shows the footprint of the ribozyme substrate strand in the docked ribozyme complex, compared to the cleavage of the substrate strand in free solution. The protection of the substrate strand in the folded ribozyme structure (which produces a solvent inaccessible core) is in good agreement with the solvent accessibility model for the hairpin ribozyme-substrate complex (Earnshaw et al. (1997) J. Mol. Biol. 274:197-212).

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein. All references referred to herein, including any patent, patent application or non-patent publication, are hereby incorporated by reference in their entirety.

The invention claimed is:

1. A method for analyzing the structural properties of an RNA molecule comprising:
(a) contacting said RNA molecule with a cleavage reagent capable of partially hydrolyzing said RNA molecule, wherein said partial hydrolysis is attenuated in a region of said RNA molecule that is relatively inaccessible to solvent; and
(b) separating and detecting the cleaved RNA by IP-RP-HPLC, wherein the absence of cleavage events in a region of the RNA indicates that said region is relatively inaccessible to solvent.

2. The method of claim 1, wherein said IP-RP-HPLC employs a separation medium that is substantially free of multivalent cations that are capable of interfering with polynucleotide separations.

3. The method of claim 2, wherein said separation medium comprises particles selected from the group consisting of silica, silica carbide, silica nitrite, titanium oxide, aluminum oxide, zirconium oxide, carbon, insoluble polysaccharide, and diatomaceous earth, the particles having separation surfaces which are coated with a hydrocarbon or non-polar hydrocarbon substituted polymer, or have substantially all polar groups reacted with a non-polar hydrocarbon or substituted hydrocarbon group, wherein said surfaces are non-polar.

4. The method of claim 2, wherein said separation medium comprises polymer beads having an average diameter of 0.5 to 100 microns, said beads being unsubstituted polymer beads or polymer beads substituted with a moiety selected from the group consisting of hydrocarbon having from one to 1,000,000 carbons.

5. The method of claim 4, wherein said beads are substituted with a moiety selected from the group consisting of methyl, ethyl, or hydrocarbon having from 23 to 1,000,000 carbons.

6. The method of claim 2, wherein said separation medium comprises a monolith.

7. The method of claim 2, wherein said separation medium has been subjected to acid wash treatment to remove any residual surface metal contaminants.

8. The method of claim 2, wherein said separation medium has been subjected to treatment with a multivalent cation binding agent.

9. The method of claim 2, wherein said IP-RP-HPLC employs a mobile phase comprising a solvent selected from the group consisting of alcohol, nitrile, dimethylformamide, tetrahydrofuran, ester, ether, and mixtures of one or more thereof.

10. The method of claim 9, wherein said mobile phase comprises acetonitrile.

11. The method of claim 2, wherein said mobile phase comprises a counterion agent selected from the group consisting of lower alkyl primary amine, lower alkyl secondary amine, lower alkyl tertiary amine, lower trialkylammonium salt, quaternary ammonium salt, and mixtures of one or more thereof.

12. The method of claim 11, wherein said counterion agent is selected from the group consisting of octylammonium acetate, octadimethylammonium acetate, decylammonium acetate, octadecylammonium acetate, pyridiniumammonium acetate, cyclohexylammonium acetate, diethylammonium acetate, propylethylammonium acetate, propyldiethylammonium acetate, butylethylammonium acetate, methylhexylammonium acetate, tetramethylammonium acetate, tetraethylammonium acetate, tetrapropylammonium acetate, tetrabutylammonium acetate, dimethydiethylammonium acetate, triethylammonium acetate, tripropylammonium acetate, tributylammonium acetate, tetrapropylammonium acetate, tetrabutylammonium acetate, triethylammonium hexafluoroisopropyl alcohol, and mixtures of one or more thereof.

13. The method of claim 12, wherein said counterion agent is tetrabutylammonium acetate.

14. The method of claim 12, wherein said counterion agent is triethylammonium acetate.

15. The method of claim 11, wherein said counterion agent includes an anion selected from the group consisting of acetate, carbonate, phosphate, sulfate, nitrate, propionate, formate, chloride, and bromide.

16. The method of claim 2, wherein said detection is achieved using Matched Ion Polynucleotide Chromatography.

17. The method of claim 2, wherein said RNA molecule is detectably labeled.

18. The method of claim 17, wherein said detectable label is fluorescent.

19. The method of claim 118, wherein said detectable label is selected from the group consisting of FAM, JOE, TAMRA, ROX, HEX, TET, Cy3, and Cy5.

20. The method of claim 19, wherein said detectable label is FAM.

21. The method of claim 2, wherein said cleavage reagent is a hydroxyl radical.

22. The method of claim 21, wherein said hydroxyl radical is generated using Fe(EDTA)^{2-.}

23. The method of claim 2, wherein said cleavage reagent is a nuclease.

24. The method of claim 23, wherein said nuclease is an RNase.

25. The method of claim 2, wherein said IP-RP-HPLC separation is phased by running a parallel RNA cleavage reaction.

26. The method of claim 25, wherein said DNA cleavage reaction is an RNA sequencing reaction.

27. The method of claim 2, wherein said RNA molecule includes region that is relatively inaccessible to solvent owing to intramolecular interactions.

28. The method of claim 2, wherein said method is used to characterize the three-dimensional structure of said RNA molecule.

29. The method of claim 2, wherein said RNA molecule includes region that is relatively inaccessible to solvent owing to intermolecular interactions.

30. The method of claim 2, wherein said RNA molecule is a ribozyme, and wherein said method is used to characterize the interaction of said ribozyme with a substrate.

31. The method of claim 29, wherein said intermolecular interaction is between said RNA molecule and an RNA-binding protein.


SEQUENCE LISTING (3 sequences)

SEQ ID NO: 1 - RNA, 13 nt, Artificial Sequence
  oligoribonucleotide
ucgcaguccuauu

SEQ ID NO: 2 - RNA, 21 nt, Artificial Sequence
  oligoribonucleotide
ggcgugguacauuaccuggua

SEQ ID NO: 3 - RNA, 32 nt, Artificial Sequence
  oligoribonucleotide
aaauagagaagcgaaccagagaaacacacgcc